(19) United States
(12) Patent Application Publication
(10) Pub. No.: US 2006/0247184 A1
(43) Pub. Date: Nov. 2, 2006

(54) Treatment and prophylaxis of diseases and infections pigs and poultry

(75) Inventor: Michael John Sanders, London (GB)

Correspondence Address: HOWSON AND HOWSON, SUITE 210, 501 OFFICE CENTER DRIVE, FT WASHINGTON, PA, 19034, US

(73) Assignee: Eco Animal Health Ltd., London (GB)

(21) Appl. No.: 11/479,721

(22) Filed: Jun. 30, 2006

Related U.S. Application Data
(63) Continuation of application No. 10/398,086, filed on Mar. 31, 2003.

(30) Foreign Application Priority Data
Oct. 18, 2000 (GB) 0025556.2

(51) Int. Cl. A61K 31/7048

(52) U.S. Cl. 514/28

(57) ABSTRACT

The use of AIVLOSIN, as such or as a pharmaceutically acceptable (non-toxic) derivative such as an acid addition salt, for the preparation of a veterinary medicament for the treatment and prophylaxis of diseases and infections of pigs and poultry. In particular, the diseases and infections treatable are necrotic enteritis in poultry and Lawsonia infections, Mycoplasma diseases, and swine dysentery in pigs.

### CROSS-REFERENCE TO RELATED APPLICATIONS

This application is a continuation of U.S. patent application Ser. No. 10/398,086, filed on Mar. 31, 2003, which is the national phase filing of PCT/GB01/04575, filed on Oct. 15, 2001, which claims priority to GB 0025556.2, filed on Oct. 18, 2000.

### BACKGROUND OF INVENTION

The present invention relates to the use of antibiotics as veterinary medicaments for the treatment or prophylaxis of diseases and infections of animals, specifically pigs and poultry.

Pigs and poultry, especially those which are intensively reared or reared in large-scale operations, have tendency to suffer from or risk catching a variety of diseases and infections, for example, Mycoplasma diseases in pigs and poultry, Lawsonia infections and swine dysentery in pigs and necrotic enteritis in poultry. Medicaments have been proposed or used for the treatment of individual diseases or infections of these types. Such medicaments are either not in general thought to be highly effective in a wide range of diseases or infections or not thought to be effective at low dosage levels. Thus, for example, tiamulin, which is used to treat swine dysentery, is not effective in Lawsonia and not very effective against Mycoplasma diseases and erythromycin, which is used against Mycoplasma has no reported effect against swine dysentery or Lawsonia.

### DESCRIPTION OF THE INVENTION

Surprisingly, we have now found that the known antibiotic AIVLOSIN (otherwise known as 3-O-acetyl-4″-O-isovaleryl-tylosin), which has previously been used in high doses for the treatment and control of Mycoplasma diseases in poultry, is also effective in the prevention and treatment of Lawsonia infections (ileitis) and swine dysentery in pigs and the prevention and treatment of necrotic enteritis in poultry. It is also effective in the treatment and control of Mycoplasma diseases in pigs and at much lower doses than hitherto used, in the treatment and control of Mycoplasma diseases in poultry. Furthermore, when used in combination with tetracyclines; particularly chlortetracycline or oxytetracycline, synergistic results have been found to occur.

The present invention therefore provides for the use of AIVLOSIN, as such or as a pharmacologically acceptable (non-toxic) derivative such as an acid addition salt, in the preparation of a veterinary medicament for the treatment or prophylaxis of Lawsonia infections or swine dysentery in pigs or necrotic enteritis in poultry, as well as a process for the treatment or control of Lawsonia infections or swine dysentery in pigs, or necrotic enteritis in poultry comprising administering to pigs or poultry, as the case may be, an effective amount of AIVLOSIN or a pharmacologically effective derivative thereof.

It includes the use of AIVLOSIN, as such or as a pharmacologically acceptable derivative, in the preparation of a veterinary medicament for the treatment or control of Mycoplasma diseases in pigs and poultry, the medicament being added to food at a level of less than 200 ppm (200 g/1000 kg of feed), as well as the corresponding process for treatment or control of Mycoplasma diseases in pigs and poultry.

The invention also includes a veterinary medicament comprising as active ingredients in admixture AIVLOSIN and a tetracycline, especially chlortetracycline or oxytetracycline.

It also includes a tablet coated composition for addition to animal feed (e.g. for pigs or poultry) comprising AIVLOSIN in particulate form coated with polyvinyl pyrrolidone.

In British Patent Specification No. 1,539;907 there are disclosed tylosin derivatives having acyl groups in the 3 and 4″ positions and acid addition salts thereof, specifically the tartaric, acetic, propionic, citric, succinic, hydrochloric, sulphuric and phosphoric acid addition salts. Amongst the tylosin derivatives specifically disclosed there is 3-O-acetyl-4″-O-isovaleryl-tylosin, which is now commonly known as AIVLOSIN. This compound has the formula: 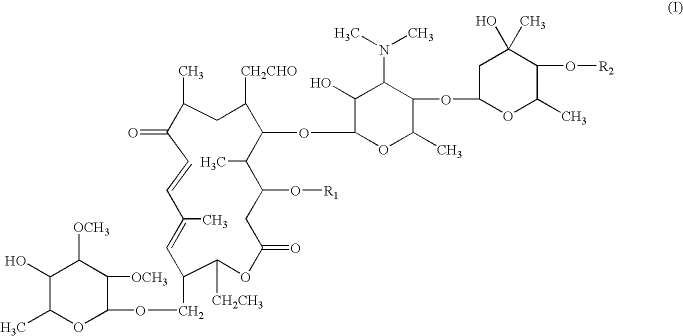
where R₁ is acetyl and R₂ is isovaleryl. There is also disclosed a process for the production of AIVLOSIN by the biochemical acylation of tylosin or an appropriately partially acylated tylosin by means of an appropriate acylating microorganism of the genus Streptomyces, especially one selected from Streptomyces thermotolerans (ATCC 11416), Streptomyces fungicidus subsp. espinomyceticus (ATCC 21574), Streptomyces mycarofaciens (ATCC 21454) and Streptomyces hygroscopicus (ATCC 21582), in the presence of the appropriate acyl donor, especially acetyl COA, isovaleryl CoA, acetic acid, isovaleric acid, potassium, sodium or ammonium salts of those acids, methanol and ethanol esters of these acids, amides of these acids and α-oxovaleric acid.

The said Specification mentions that the tylosin derivatives can be administered to humans or animals and refers to their activity against a number of gram-positive bacteria, including some drug-resistant bacteria, but it does not specifically refer to the use of the derivatives in the treatment or control of specific diseases or infections of animals, although it does say that they can be employed on humans, livestock, household pets, laboratory animals and poultry and in the enteral, parenteral or topical control of infectious diseases in a similar manner as for known macrolide antibiotic drugs.

In fact, AIVLOSIN on the basis of its initial Japanese marketing registration (No 4 chika AC1771) has to date been marketed and approved for marketing only for the treatment and control of Mycoplasma diseases in pigs and poultry at high doses of 200 to 500 ppm in feed. There should be no reason to suppose that it would be suitable for the treatment and prophylaxis of other infections and diseases of pigs and poultry, and in particular other macrolide antibiotics having an effectiveness against Mycoplasma diseases such as erythromycin do not have any effect or any significant effect against other infections of pigs and poultry such as those mentioned above. It is, of course, a feature of the approvals schemes which apply in all major countries that a veterinary medicament which is approved for marketing for one specific purpose cannot be marketed or recommended for use for any other specific purpose without a separate authorization or approval from the relevant Authority. There is thus a strong counter-incentive to the use of even known antibiotics for new veterinary uses.

However, we have now found and confirmed from extensive in vitro and in vivo (animal) trial work that AIVLOSIN and acceptable derivatives thereof are effective in the prevention and treatment of Lawsonia infections and swine dysentery (caused by Brachyspira hyodysenteriae) in pigs at reasonable dose rates. Extensive in vitro and in vivo (animal) trial work has also found and confirmed that they are effective at low doses in the prevention and treatment of Mycoplasma disease in poultry and pigs. Finally, in vitro trial work has indicated that they are likely to be effective in the prevention and treatment of necrotic enteritis (caused by Clostridium perfringens) in poultry.

AIVLOSIN is available in free form as a white crystalline powder having a melting point of 180°-184° C., soluble in lower alcohols such as ethanol, ketones such as acetone, ethers such as diethyl ether, esters such as ethyl acetate and aromatic hydrocarbons such as toluene, although it is barely soluble in n-hexane and petroleum ether. It is very soluble in aqueous solutions of pH around and below 7 but less soluble in aqueous solutions of higher pH. Because it is a basic compound it forms acid addition salts, and the use of such salts which are pharmacologically acceptable is also included within the present invention. Acids to form acceptable acid addition salts include inorganic acids such as hydrochloric, sulphuric or phosphoric acid and organic acids such as tartaric, acetic, propionic, citric and succinic acids. Specific examples of acceptable derivatives are AIVLOSIN hydrochloride (melting point 129-133° C.) and AIVLOSIN tartrate (melting point 119-122° C.). Such derivatives are frequently more water-soluble than AIVLOSIN itself and their use may therefore have formulation advantages.

AIVLOSIN and appropriate derivatives can be formulated according to the present invention into veterinary medicaments in known ways, for example to provide compositions for oral, enteral or parenteral administration, by admixing with appropriate solid or liquid carriers and excipients for the administration route desired. Conventional ingredients can be used as carriers and excipients, for example water and salt solutions for liquid formulations and silicaceous materials-silica and silicates (such as hydrated magnesium silicate)-, cereal products (such as soybean meal and wheat flour) and other pharmacologically acceptable solids for solid formulations for oral administration. The formulations can also contain further auxiliaries and additives such as minerals, lubricants, preservatives, stabilizers, wetting agents, emulsifiers, buffers and coloring or flavoring materials in a conventional manner. In the prophylaxis or control of the diseases mentioned it is particularly convenient to include the AIVLOSIN or derivative as an additive to animal feed or drinking water for the pigs or poultry, but in the treatment of the diseases it can be included in an injectable solution, or a tablet, capsule or syrup, if desired.

AIVLOSIN (as such or in the form of an appropriate derivative, for example an acid addition salt such as the tartrate) may be formulated into premixes in various potencies from 1 to 10 by weight. A particularly suitable composition for producing such premixes comprises AIVLOSIN salt, filler such as soybean powder and additives such as hydroxypropyl cellulose and has a potency of 180 to 220 mg/g.

In order to ensure stability of AIVLOSIN in animal feed which may have been subjected to high-temperature processing for pelleted or extruded feed it is desirable to provide a coated AIVLOSIN (as such or in the form of an appropriate derivative, for example an acid addition salt such as the tartrate) in particulate form coated with polyvinyl pyrrolidone. Suitable proportions by weight are in the range active ingredient: polyvinyl pyrrolidone 50:1 to 1:1. Inert fillers and other ingredients may be present in such compositions, the overall polyvinyl pyrrolidone concentration being preferably 0.1 to 10% by weight.

The veterinary medicament formulations can also contain further active ingredients useful in the treatment of infections and diseases of pigs and poultry, such as further antibiotics, in particular tetracycline antibiotics, for that purpose. We have found from sensitivity tests that the use of AIVLOSIN together with a tetracycline antibiotic enables lower dosages of both antibiotics to be used than would be possible with either antibiotic alone in order to achieve comparable results.

The veterinary medicament formulations for use either as feed additives or as directly administered preparations may contain any convenient proportion of AIVLOSIN for example from 1% or less to 90% or more, by weight. Liquid formulations typically contain 50 to 90% by weight, whereas solid formulations typically contain 1 to 25% by weight.

For treatment or control of Lawsonia infections in pigs they may for example be administered in feed at a rate of 40 to 120 ppm by weight (40-120 g per 1,000 kg of feed) for a period of time long enough to control or treat the disease successfully, for example 7 to 14 days. For example, figures of 40 to 100 or 50 to 80 ppm may be used. A rate of 50 ppm for 10 days is usually effective in controlling the disease and a rate of 100 ppm for 10 days, is usually very successful in treating it. For treatment or control of swine dysentery comparable rates and periods may be used; administration in feed at a rate of 50 ppm for 10 days is likely to be effective in preventing an outbreak. Similar or lower rates and times are also expected to be effective when AIVLOSIN or a derivative is used in the treatment or prophylaxis of necrotic enteritis.

For control of Mycoplasma disease in young poultry an AIVLOSIN formulation can be injected directly into eggs. This also makes day-old chicks negative to pleuro-pneumonia-like organisms (PPLO).

The rates of administration and periods for which administration is made to treat or control Mycoplasma in poultry and pigs are surprisingly low. The original Japanese marketing registration referred to levels of 200 to 400 ppm by weight in feed whereas less than 200 ppm is used in the present invention, preferably 40-150 ppm or less or example the ranges mentioned above.

When AIVLOSIN or a derivative is directly administered for treatment or control of Lawsonia infections, swine dysentery or necrotic enteritis the administration levels based on body weight may be in the range 1 to 8, preferably 1 to 5 mg/kg body weight/day.

When AIVLOSIN is used in admixture with tetracycline, especially chlortetracycline or oxytetracycline for synergistic results against Brachyspira hyodysenteriae or Mycoplasma synoviae, the amounts of each ingredient may be reduced substantially, for example to one half to one third, of the amount of the same ingredient used alone. The mixtures may contain AIVLOSIN and the tetracycline in a wide range of weight ratios, for example 10 parts or less of tetracycline per part of AIVLOSIN by weight, especially 10:1 to 5:1 or 8:1 to 6:1 by weight.

The following Examples, in which parts are by weight, illustrate the use of AIVLOSIN in the manufacture of veterinary medicaments or preparations for treatment or prophylaxis of the pig and poultry infections according to the present invention and the synergistic effect.

### EXAMPLE 1

20 parts of AIVLOSIN API (active pharmaceutical ingredient) made into a solution in water is mixed with 80 parts of soybean meal, and the mixture is spray dried to give a solid additive for feedstuff containing 200 kg AIVLOSIN activity per 1000 kg. This formulation can be added to pig and poultry feed to provide an in-feed concentration of AIVLOSIN of 25 to 200 g AIVLOSIN per 1000 kg final feed.

### EXAMPLE 2

25 parts of AIVLOSIN 20% is mixed with 50 parts of hydrated magnesium silicate (an inert silica), 24 parts of wheat feed flour and 1 part of liquid paraffin EP as a powder blend to give a solid additive for feedstuff containing 50 kg AIVLOSIN activity per 1000 kg. This formulation can be used in pig and poultry feed as in Example 1.

### EXAMPLE 3

5 parts of AIVLOSIN 20% as used in Example 2 is mixed with 40 parts of hydrated magnesium silicate, 54 parts of wheat feed flour and 1 part of liquid paraffin EP as a powder blend to give a solid additive for feedstuff containing 10 kg AIVLOSIN activity per 1000 kg. This formulation can be used in pig and poultry feed as in Example 1.

### EXAMPLE 4

AIVLOSIN is dissolved in water to provide an aqueous solution containing 80-90% AIVLOSIN activity for use in drinking water for pigs or poultry. This formulation can be added to drinking water to provide AIVLOSIN concentrations in drinking water in the range 25 to 100 g per 200 liters of drinking water.

### EXAMPLE 5

AIVLOSIN API containing more than 80% w/w AIVLOSIN tartrate was mixed into an 850 kg batch comprising:

| | | |
|---|---|---|
| AIVLOSIN API | 163-169 | kg |
| Hydroxypropyl cellulose, Ph. Eur. | 8.2-8.5 | kg |
| Water, Ph. Eur. | 800-1200 | liters |
| Non-fat soybean powder | 720 | kg |

The batch was processed and the water was removed during processing. The input of AIVLOSIN API was adjusted for content value of free base, determined by HPLC, of the raw material to achieve a final product bioassay potency of 180-220 mg/g. The product (AIVLOSIN FG 200), which could also be produced in other batch sizes, was suitable for manufacturing AIVLOSIN premixes in various potencies from 1% to 10%.

### EXAMPLE 6

Coated AIVLOSIN formulations possessing stability in animal feed after high-temperature processing for pelleted or extruded feed were produced in batches of 1000 kg (although other batch sizes could be used) from the following ingredients:

| | | |
|---|---|---|
| AIVLOSIN FG 200 (see Example 5) | 250.0 | kg |
| Paraffin, Light Liquid, Ph. Eur. | 10.0 | kg |
| Wheat feed flour | 240.0 | kg |
| Polyvinyl pyrrolidone | 10.0-100.0 | kg |
| Sepiolite | to 1000.0 | kg |

Trial Results
1. Lawsonia Infections.

Lawsonia infections (ileitis or proliferative porcine enteropathy) in pigs are caused by the pathogen Lawsonia intracellularis, which was isolated only some six years ago and is a bacteria residing in the cells of the intestinal wall of the lower small intestine of pigs. To date, few antimicrobials have been recognized as effective in preventing and treating the disease, which is widespread throughout the world in its incidence and is of considerable economic importance in pig rearing and breeding. Extensive trial work by us both in vitro and in vivo on pigs have shown that AIVLOSIN is very effective in treating the disease and preventing it from spreading further in an infected environment. The following Table 1 shows clinical results of an AIVLOSIN porcine proliferative enteropathy efficacy study carried out by us.

TABLE 1

| | Mortality | Lesion | Lesion |
|---|---|---|---|
| | Rate | Incidence | Severity |
| Group | (%)&[n] | (%) | (ins.) |
| Control | 15.1 [3] | 80.0 | 43.1 |
| Aivlosin 50 ppm | 13.3 [4] | 73.3 | 36.2 |
| Aivlosin 100 ppm | Nil | 33.3* | 3.15* |

(*statistically significant (p-value < 0.001) from other 10 groups)

There was also considerable improvement in feed intake, weight gain and feed efficiency in treatment groups with AIVLOSIN. For all these production parameters AIVLOSIN performed well.

AIVLOSIN at an inclusion rate of 50 grams per tonne (1000 kg) of feed (50 ppm), provided for 10 days was effective in controlling the disease, while at 100 ppm for 10 days the outbreak was very successfully treated.

2. Swine Dysentery.

Swine dysentery is caused by Brachyspira hyodysenteriae, a bacteria which resides in the lumen of the large intestine of pigs, where it hides in the crypts and feeds on the mucosa. It was formerly treated with nitroimidazoles but these are now banned from use in animals destined for human consumption. Chemicals from the pleuromutilin group, especially tiamulin, are available for treatment of the disease, and tylosin has been suggested for use in the past, although use has decreased dramatically in recent years due to development of tylosin resistance by the pathogen.

AIVLOSIN has been tested in vitro and in vivo by us for its effectiveness against the bacteria as compared with tiamulin and tylosin. In vitro test results are shown in Table 2 below (MIC referring to minimum inhibitory concentration, namely the lowest concentration in mcg/ml of active ingredient which inhibits growth of the Brachyspira hyodysenteriae strain under investigation.

TABLE 2

| | MIC Aivlosin | MIC Tylosin |
|---|---|---|
| Strain | mcg/ml | mcg/ml |
| PO268-07.98 | 12.5 | >200 |
| AF 6/80 | 1.55 | 6.25 |
| PI8A | 12.5 | >200 |

It is apparent that AIVLOSIN is far more effective in vitro than tylosin.

Testing by us in an animal disease model, where non-infected pigs were artificially infected with a virulent strain of bacteria, showed AIVLOSIN to be effective in preventing an outbreak when given at 50 ppm in the feed for 10 days, whereas tylosin needed to be provided for a period of 21 days or longer, whilst tiamulin needs to be provided for the whole period in which the pigs are at risk.

In treating the disease AIVLOSIN at 50 ppm performed better than or equal to both tiamulin (in results) and tylosin (in duration).

Prevention of Clinical Disease

Group 1: unmedicated challenged group.
Group 2: medicated with AIVLOSIN 50 ppm for 10 days.

Group 3: unchallenged unmedicated.

RESULTS OVER THE TREATMENT PERIOD

| | | | Feed |
|---|---|---|---|
| | Clinical | AV. Daily | conversion |
| | disease | weight gain | ratio |
| Group 1 | 45% | not done | not done |
| Group 2 | 0 | 614 g. | 1.14 |
| Group 3 | 0 | 646 g. | 1.18 |

TREATMENT OF CLINICAL OUTBREAK (Mean score = mean
clinical scoring: 0 = normal, 6 = moribund)

| | Mean | | Mean days |
|---|---|---|---|
| | score at | Mean days | to clear |
| Treatment Group | start | to recovery | B.Hyo |
| Tiamulin 100 ppm | 4.0 | 4.4 | 5.8 |
| 10 days | | | |
| Tylosin 100 ppm | 3.7 | 2.1 | 3.0 |
| 21 days | | | |
| Aivlosin 50 ppm | 3.9 | 2.5 | 3.0 |
| 10 days | | | |
| Aivlosin 100 ppm | 3.9 | 2.3 | 3.0 |
| 10 days | | | |

3. Necrotic Enteritis.

Necrotic enteritis is a disease caused by toxins produced by the bacteria Clostridium perfringens, which can lead to widespread destruction of the gut lining with consequent increases in mortality and low growth rates. Virginiamycin and zinc bacitracin have been used in the past as growth promoters to control this disease but they have recently been banned for use. There has been no previous proposal to use macrolide antibiotics to treat or control necrotic enteritis, nor is there any expectation that such antibiotics would be useful.

However, in vitro tests carried out by us show that, as can be seen from Table 3 below, AIVLOSIN is significantly more effective than zinc bacitracin against various strains of Clostridium perfringens.

TABLE 3

| | | MIC Zinc |
|---|---|---|
| | MIC Aivlosin | Bacitracin |
| Strain | (mcg/ml) | (mcg/ml) |
| 410 | 0.078 | 3.125 |
| 413 | 0.039 | 6.25 |
| 412 | 0.039 | 6.25 |
| 395 | 0.039 | 3.125 |
| 378 | 0.039 | 3.125 |
| 389 | 0.039 | 1.56 |
| 392 | 0.039 | 1.56 |

4. Sensitivity of Bacteria for AIVLOSIN and Tetracyclines, Alone and in Combination.

Sensitivity is expressed as the lowest concentration of an antibiotic (in mcg/ml) that inhibits the growth of the test bacteria (MIC=Minimum Inhibitory Concentration).

Brachyspira Hyodysenteriae

COMBINATION OF AIVLOSIN (AIV) AND
CHLORTETRACYCLINE (CTC)

| | | |
|---|---|---|
| | | MIC of each for |
| | | combination of |
| MIC CTC | MIC AIV | CTC + AIV (mcg/ml) |

| Strain | (mcg/ml) | (mcg/ml) | CTC | AIV |
|---|---|---|---|---|
| P265-9-97 | 16 | 50 | 4.0 | 6.25 |
| P578-6-97 | 16 | 50 | 8.0 | 12.5 |
| P268-9-97 | 16 | 25 | 8.0 | 1.55 |
| AF6 | 0.5 | 3.1 | 0.125 | 0.75 |

Mycoplasma Synoviae

COMBINATION OF AIVLOSIN AND
CHLORTETRACYCLINE

| | | |
|---|---|---|
| | | MIC of each for |
| | | combination |
| MIC CTC | MIC AIV | CTC + AIV (mcg/ml) |

| Strain | (mcg/ml) | (mcg/ml) | CTC | AIV |
|---|---|---|---|---|
| 173 | 0.78 | 0.031 | 0.1 | 0.015 |
| 185 | 0.78 | 0.031 | 0.1 | 0.015 |
| 211 | 0.39 | 0.062 | 0.1 | 0.031 |
| 312 | 3.125 | 0.062 | 0.1 | 0.031 |
| wvu 1835 | 0.39 | 0.031 | 0.1 | 0.015 |

COMBINATION OF AIVLOSIN AND OXYTETRACYCLINE (OTC)

| | | |
|---|---|---|
| | | MIC of each for |
| | | combination |
| MIC OTC | MIC AIV | OTC + AIV (mcg/ml) |

| Strain | (mcg/ml) | (mcg/ml) | OTC | AIV |
|---|---|---|---|---|
| 173 | 0.39 | 0.031 | 0.1 | 0.015 |
| 185 | 0.20 | 0.031 | 0.1 | 0.015 |
| 211 | 0.39 | 0.062 | 0.1 | 0.031 |
| 312 | 0.78 | 0.062 | 0.1 | 0.031 |
| wvu 1835 | 0.39 | 0.031 | 0.1 | 0.015 |

5. AIVLOSIN used as an Injection Directly into Eggs in Order to Prevent Mycoplasma Disease in Young Chickens. Also to Make Day Old Chicks PPLO (Pleuro-Pneumonia-Like Organisms) Negative.

The following work on AIV was conducted using the EMBREX IN OVO injection system.

The absorption of AIVLOSIN after oral intake is 150-200% better compared to tylosin, and penetration to target site is better. MIC compared to tylosin is improved.

The AIVLOSIN injection was prepared by adding 40 g AIVLOSIN activity using water soluble AIVLOSIN to 1 liter of sterile saline.

The following trial was carried out for the purpose of studying the effect of AIVLOSIN injected in ovo, against Mycoplasma for chicken and turkeys.

A pilot trial was conducted to test toxicity of several antibiotics injected in ovo at 18 days embryonated eggs. AIVLOSIN was tested at 1, 3 and 5 mg per egg (0.05 ml), 25 eggs per group. Results were very good.

% Hatchability:

100% at 1 mg

95.8% (1 embryo alive but not pipped) at 3 mg

100% at 5 mg.

### CONCLUSIONS

Injection with AIVLOSIN (4 mg per egg in 0.05 ml saline) did not influence hatchability in the treatment group.

Injection with saline (0.05 ml per egg) did not influence hatchability in the control group.

| | | hatched | hatched | hatched |
|---|---|---|---|---|
| | | chickens | chickens | chickens |
| HATCHABILITY | | 15 hours | 19 hours | 20 hours |
| (Gemonde | Injection** | post Inj | post Inj | post Inj |
| TREATMENT (10 | 449 hours | 8 | 10 | 10 |
| eggs) | | | | |
| CONTROL (10 | 449 hours | 6 | 9 | 10 |
| eggs) | | | | |

**all aircells still intact (candled before injection); weight of chickens 3 days after hatch (2 days after hatch in hatchery).

| | Female Chickens | Male Chickens |
|---|---|---|
| | 67 | 51 |
| | 66 | 69 |
| | 54 | 66 |
| | 65 | 57 |
| | 70 | 65 |
| | 61 | 60 |
| | | 61 |
| | | 66 |
| | | 60 |
| | | 61 |
| | | 63 |
| | | 63 |
| | | 50 |
| total | 383 | 792 |
| Average | 63.8 | 60.9 |

What is claimed:

1. A method for the treatment, control or prophylaxis of Lawsonia infections in pigs comprising the step of administering to a pig in need thereof an effective amount of a compound selected from 3-O-acetyl-4″-O-isovaleryl-tylosin and salts thereof.

2. The method of claim 1, wherein said compound selected from 3-O-acetyl-4″-O-isovaleryl-tylosin and salts thereof is administered to said pig as a pharmacologically acceptable formulation in a mode selected from addition to the pig's feed and addition to the pig's drinking water.

3. A method for the treatment, control or prophylaxis of necrotic enteritis in poultry comprising the step of administering to poultry in need thereof an effective amount of a compound selected from 3-O-acetyl-4″-O-isovaleryl-tylosin and salts thereof.

4. The method of claim 3, wherein said compound selected from 3-O-acetyl-4″-O-isovaleryl-tylosin and salts thereof is administered to said poultry as a pharmacologically acceptable formulation in a mode selected from addition to the poultry's feed and addition to the poultry's drinking water.

5. A method for the treatment, control or prophylaxis of Mycoplasma diseases in pigs and poultry, comprising the step of administering to a pig or to poultry in need thereof a compound selected from 3-O-acetyl-4″-O-isovaleryl-tylosin and salts thereof, said compound being in the pig's or the poultry's feed in an amount in the range 40 to 150 ppm by weight.

6. A method for the treatment, control or prophylaxis of swine dysentery in pigs, comprising the step of administering to a pig in need thereof a compound selected from 3-O-acetyl-4″-O-isovaleryl-tylosin and salts thereof.

7. The method of claim 6, wherein said compound selected from 3-O-acetyl-4″-O-isovaleryl-tylosin and salts thereof is administered to said pig in a pharmacologically acceptable formulation in a mode selected from addition to the pig's feed and addition to the pig's drinking water.

8. A method for making poultry negative to pleuro-pneumonia like organisms (PPLO), comprising the step of administering to said poultry a compound selected from 3-O-acetyl-4″-O-isovaleryl-tylosin and salts thereof.

9. The method of claim 8, wherein said compound is administered by injection in a pharmacologically acceptable formulation.

10. The method of claim 9, wherein said compound is administered by injection directly into eggs.

11. A feed for poultry or pigs, containing 40 to 150 ppm by weight of a compound selected from 3-O-acetyl-4″-O-isovaleryl-tylosin and salts thereof.

12. A stable coated composition for addition to animal feed comprising a compound selected from 3-O-acetyl-4″-O-isovaleryl-tylosin and salts thereof in particulate form, coated with polyvinyl pyrrolidone.

13. A veterinary medicament comprising as active ingredients in admixture:
a compound selected from 3-O-acetyl-4″-O-isovaleryl-tylosin and salts thereof; and
a tetracycline.

14. The veterinary medicament of claim 13, wherein the tetracycline is selected from chlortetracycline and oxytetracycline.

15. The veterinary medicament of claim 13, containing the active ingredients in a weight ratio in the range 1:5 to 1:10.
